(19) United States
(12) Patent Application Publication
(10) Pub. No.: US 2005/0089551 A1
(43) Pub. Date: Apr. 28, 2005

(54) Clotting agent-containing window dressing

(76) Inventor: Elizabeth A. Recupero, Lambertville, NJ (US)

Correspondence Address: Kenneth P. Glynn, Esq.;Glynn & Associates, P.C., 24 Mine Street, Flemington, NJ, 08822, US

(21) Appl. No.: 10/690,792

(22) Filed: Oct. 22, 2003

(51) Int. Cl. A61K 9/70; B32B 27/04; B32B 27/12

(52) U.S. Cl. 424/443; 442/123

(57) ABSTRACT

A window dressing has a self-contained, ready to use blood clotting agent. It includes a top element, a middle element, a bottom element, and at least one clotting agent located on the bottom of the middle element. The top element has a top and a bottom and is of a predetermined shape with a first perimeter distance. By “perimeter distance” is meant the measured perimeter. The top element has a central cut-out area with a second predetermined distance, the first perimeter distance being greater than said second perimeter distance, so as to establish a window opening. The middle element has a top and a bottom, and is a clear polymer window with a predetermined size, shape, and window perimeter distance greater than said second perimeter distance of the top element so that it will fit into the window opening of the top element and overlap the inside edges of it. This middle element is the window and is attached to the bottom of the top element. The bottom element has a top and a bottom and has a central cut out area. The top of the bottom element is attached to one of the top element and the middle element or both, at their bottom(s.). In preferred embodiments, all elements are sterile and the middle element is a water impermeable clear polymer material that acts both as a window and as a blood containment layer. The clotting is agent attached to the bottom of the middle element and within the cut out of the bottom element.

### BACKGROUND OF THE INVENTION

1. Field of the Invention

The present invention relates to devices for the treatment of bleeding that may result from insertion and subsequent removal of intrusive devices to the body. More specifically, the present invention is a window dressing that contains a clotting agent on the inside of the window to permit a user to apply the clotting agent where and when it is needed.

2. Information Disclosure Statement

The following patents relate to wound dressings or blood clotting:

U.S. Pat. No. 2,163,588 describes a hemorrhage arrestor adapted to be pressed into an open bleeding wound for stopping the flow of blood and comprising, a soft resilient compressible porous body of blood repellant material being self retaining and resilient when subjected to blood, and a water insoluble viscous grease carried by said body in a proportion effective to substantially fill the pores of said body on compression thereof into a wound at normal applying pressures to provide a substantially blood impervious plug in the wound.

U.S. Pat. No. 2,688,586 describes a method of making a surgical dressing having improved stability and hemostatic properties which comprises adding to a surgical dressing containing alginate material, a minor amount, between about 0.5% and about 10% by weight dry basis, based on said alginate material, of an acid of the group consisting of citric, maleic, malic, nitric, phosphoric and tartaric acids in solution, the amount of said acid being in excess of that required to convert the alginate material to alginate salt content below a value corresponding with 3.0% calcium, and thereafter drying said dressing without removing unreacted acid nor reaction products.

U.S. Pat. No. 2,772,999 describes a surgical composition for coagulating blood containing a hemostatic amount, at least about 2% of cellulose derivative of the group consisting of free acid cellulose derivative of the group, consisting of free acid cellulose glycolic acid ether and free acid cellulose hydroxypropionic acid ether having degree of substitution at least about 0.5, and degree of neutralization in the approximate range 0 to 60% but sufficiently low so that the free carboxy content of the cellulose is at least 0.5 per glucose unit.

U.S. Pat. No. 2,773,000 describes a surgical dressing having adjacent a wound-contacting external surface thereof, a non-toxic hydrophilic aliphatic poly-ol compound normally existing in non-crystalline condition and in amount sufficient to produce easy removal of said dressing from a clot of blood, said dressing also containing cellulose derivative of the group consisting of free acid cellulose glycolic acid ether and free acid cellulose hydroxypropionic acid ether in hemostatic amount at least about 0.30 mg. per square inch of said surface, said cellulose derivative having degree of substitution at least about 0.5 and degree of neutralization in the approximate range of 0 to 60% and sufficiently low so that the free carboxy content of the cellulose is at least 0.5 per glucose unit, said cellulose derivative further having degree of polymerization indicated by viscosity of a 0.50% by weight solution of the free acid in a 0.50 N NaOH solution at 25.50° C. of at least 5.7 seconds as determined in a modified Ostwald-Fenske viscometer pipette ASTM-D445 #300, as compared with a zero pipette reading of 5.0 seconds for a 0.50 N. NaOH solution at the same temperature.

U.S. Pat. No. 3,206,361 describes a protective wound cover comprising an absorbent base containing as a hemostatic agent methylaminoacetocatechol stabilized with citric acid and trisodium citrate.

U.S. Pat. No. 3,645,835, relates to a moisture-vapor-permeable pressure-sensitive adhesive material for use on animal skin and nails, e.g. a surgical drape, suture strip or sheet, adhesive dressing, bandage, plaster, strapping tape, decorative nail covering, or decorative cosmetic product. The adhesive material comprises a backing material and a pressure-sensitive adhesive on at least substantially the whole of the body adhering portion of at least one surface of said backing material, both said backing material and said adhesive being moisture-vapor-permeable and unaffected by water and at least one of said backing material and said adhesive comprising a synthetic polymer and being continuous and nonpermeable to liquid water, said adhesive material having a moisture vapor permeability of at least 300 g./sq. meter/24 hours/40° C./80 percent RH.

U.S. Pat. No. 4,265,233 describing a wound healing material having blood coagulation Factor XIII fixed thereto. This material promotes the formation of stabilized fibrin at a wound site and is effective for a long period of time, thereby protecting the wound and promoting its healing.

U.S. Pat. No. 4,363,319 describes a prepackaged coagulant composition and method of preparing same has been devised for use in direct or topical application to wounds so as to cause superficial clotting in which thrombin is combined with special preservatives and a saline solution. A ready-to-use bandage is saturated with the resultant solution, sealed and stored so as not to loose its reactive properties with the fibrinogen in the blood when applied to a wound or cut.

U.S. Pat. No. 4,417,981 describes a blood collection device for receiving whole blood adapted to be centrifuged into phases of lighter and heavier specific gravity includes a container with proximal and distal ends and a penetrable stopper at the proximal end. A separator assembly inside the container includes a receptacle with thixotropic barrier material therein. An aperture in the receptacle faces toward the distal end of the container. The barrier material has a specific gravity intermediate the specific gravities of the lighter and heavier phases of blood and is adapted to flow under its own influence through the aperture during centrifugation. The barrier material then travels to a position intermediate the lighter and heavier phases of blood separated during centrifugation and adheres to the inside wall of the container to form a barrier.

U.S. Pat. No. 5,266,199 relates to an apparatus used in a centrifugal separating step for sample preparation in the field of clinical chemistry. Especially, the invention relates to a separating apparatus used for isolating a serum fraction from a freshly isolated blood sample without contamination with a clot fraction. A serum separating apparatus according to the present invention is characterized by comprising an elastic body in the form of a cylindrical tube and a ball which can fitted to an opening of the elastic body and by positioning at a boundary of said fractions after the centrifugation.

U.S. Pat. No. 5,533,518 describes a blood collection assembly includes a tube, which may be evacuated, and an insert therein. The insert is descendably affixed to the tube wall and, after separating a blood sample taken in the tube into solid and liquid phases, descends during centrifugation and comes to rest at the solid-liquid interface on a projection from the tube bottom. An interior surface of the assembly may be modified to render it clot activating. The invention includes a method for preparing a blood sample for analysis using the assembly.

U.S. Pat. No. 6,569,185 B2 relates to a method of performing a vascular closure whereby closure material is safely injected into the blood vessel and into the vascular opening.

U.S. Pat. No. 6,573,419 B2 describes a compression dressing is disclosed. The compression dressing comprises a self-adhering elastic bandage strip designed for exerting a compressive force when wrapped around a body part sufficient to hold the compression dressing in place for a period of time to provide a therapeutic effect to a wound and an absorbent pad affixed to an inner side of a terminal portion or to a terminal end of the self-adhering elastic bandage strip.

Notwithstanding the prior art, the present invention is neither taught nor rendered obvious thereby.

### SUMMARY OF THE INVENTION

The present invention is a unique window dressing with a self-contained, ready to use blood clotting agent. The device includes a top element, a middle element, a bottom element, and at least one clotting agent located on the bottom of the middle element.

The top element has a top and a bottom and is of a predetermined shape with a first perimeter distance. By “perimeter distance” is meant the measured perimeter. The top element has a central cut-out area with a second predetermined distance, the first perimeter distance being greater than said second perimeter distance, so as to establish an annulus, or window opening, like a flat donut or picture frame.

The middle element has a top and a bottom, and is a clear polymer window with a predetermined size, shape, and window perimeter distance greater than said second perimeter distance so that it will fit into the annulus of the top element and overlap the inside edges of it. This middle element is the window and is attached to the bottom of the top element. In other words, the middle element is located beneath the top element and is attached to the bottom of the top element to create the window. The means used to attach the middle element to the underside or bottom of the top element may be any available means, including, but not limited to, heat bonding, gluing, adhesion, stitching, chemical bonding and combinations thereof.

The bottom element has a top and a bottom and has a central cut out area. The bottom element central cut out area and the top element central cut out area are at least partially coincidental, so that a user may see through both cut outs and the middle element window. The top of the bottom element is attached to one of said the top element and the middle element or both, at their bottom(s.). In preferred embodiments, all elements are sterile and the middle element is a water impermeable clear polymer material that acts both as a window and as a blood containment layer.

The clotting is agent attached to the bottom of the middle element and within the cut out of the bottom element. In some embodiments, the window dressing blood clotting agent is in a gel format, that is, it is in a spreadable, non-running state at room temperature. It may include one or more inert carriers, such as petroleum jelly, and it may optionally include some medication, as well. The blood clotting agent may include at least one clotting activator, and any known or to be discovered clotting agent active could be used. In some embodiments, the clotting agent activator may be selected from the group consisting of diatomaceous earth, inorganic silicates, ellagic acid, epinepherin and thromboplastin.

In most preferred embodiments, the present invention window dressing with blood clotting agent includes an adhesive on the bottom of the top element, the bottom of the bottom element or both. Alternatively, although less preferred, a user could add adhesive at the time of use to the dressing or use adhesive tape to secure it to the body.

In some embodiments, the bottom element has a bottom element perimeter distance that is less than the first perimeter distance of the top element. This maintains an exposure of the bottom of the top element, so that the bottom of the top element may have the adhesive, and the bottom element may be used as a blood flow dam around the window.

Although not essential, the bottom element may be centrally positioned relative to the top element, and is shaped to establish a bottom inside frame edge of the bottom of the top element to create the blood flow dam and to define the application frame for adhesive application during manufacture. Also, in some embodiments, the bottom element is a blood absorbing material.

In some embodiments, the clotting agent is contained within a breakable container and the breakable container is attached to the bottom of the middle element. These breakable containers could be light plastic pouches, biodegradable envelopes, or the like and may be opened by merely pressing against the container or pushing it to effect a directed flow of clotting agent under the window. For example, the breakable container may be one or more gel caps.

In other embodiments, the clotting agent may be applied to the bottom of the middle element with an additional cover, so as to sandwich the clotting agent between the bottom element and the cover. The cover could be an unattached clear plastic small sheet, or it could be a flap. This cover would prevent sticking and last of clotting agent to be a peelable protective strip. A user would push the clotting agent out from under the cover or remove the cover at the time of application.

In other embodiments, the middle element (window) may have a bubble or storage volume on its underside to create space for the clotting agent. For example, the middle element may be formed of semiflexible or ridged plastic with a bubble or dome or cube or other shape molded therein to receive the clotting agent.

For some specific applications, such as kidney dialysis, or other procedure that may cause serious hemorrhaging, the window dressing further includes at least one invasive device insertion cut out adapted to at least partially enshroud an inserted invasive device. There may also be an invasive device shroud collar for attachment around said invasive device insertion cut out. Preferably, the shroud collar is removably attached to said top element. Further, preferably, the shroud collar includes an adhesive layer for attachment around the invasive device insertion cut out. Also, there may be a removable sealing strip attached to the adhesive on the bottom of the shroud collar. Additionally, a removable sealing strip may be attached to exposed underside, i.e., the bottom of the device. Thus, it may be attached to the bottom of the bottom element, the bottom of the top element, or both. In general, the window dressing removable sealing strip may be a waterproof strip that is positioned on the window dressing to cover and seal the bottom of the middle element and the blood clotting agent. Also, in order to increase absorbency and/or decrease blood flow and minimize exposure to outside bacteria, etc., the window dressing may be made such that at least one of the top element and the bottom element is a plastic foam material.

### BRIEF DESCRIPTION OF THE DRAWINGS

The present invention should be more fully understood when the specification herein is taken in conjunction with the drawings appended hereto wherein:

FIG. 1 illustrates a top view and

FIG. 2 illustrates a front cut view of one embodiment of the present invention window dressing;

FIG. 3 and FIG. 4 show top and front cut views of an alternative embodiment present invention clotting agent-containing window dressing;

FIG. 5 illustrates a top view of yet another alternative embodiment present invention clotting agent-containing window dressing; and

FIG. 6 shows a side cut view of yet another present invention window dressing.

### DETAILED DESCRIPTION OF THE PRESENT INVENTION

The present invention relates to prevent or reduction of bleeding that occurs when intrusive treatment or accessory devices are inserted into and then removed from vessels, organs or other bleeding prone parts of the body. Such devices include, but are not limited to, sample taking devices, medical delivery devices, fluid recycling devices, fluid removal devices, IVs, flushing devices, transfusion devices, etc. Typical devices are needles, tubes, probes, stents, guidewires, connectors and the like. Of particular interest are devices used in artificial organ “machines”, such as dialysis machines for kidney disease.

Although many techniques are currently available for inhibiting bleeding, such as hemostatic bandages, sutures, butterfly bandages, gels, creams and other pressure-based, clotting based or closure-based treatments, the present invention provides a unique device that results in synergistic methods that treat bleeding instantaneously, both cutting down treatment time and increasing treatment accuracy. The present invention device provides at least three, and sometimes four separate functions in a single device. More specifically, the present invention provides a wound dressing, it also includes a window that aids the user, in the beginning, with accurate location of the intrusive device, in the procedure, by providing the window, and due to the inclusion of clotting agent on the inside of the window, enables the user to remove the intrusive device and immediately and accurately apply the clotting agent to the intrusive device removal situs. Thus, instant and accurate blood clotting treatment is provided, even before the removal of the window dressing. There is no time delay to permit early massive bleeding; there is no exposure to bacteria or other infection possibilities, and the accuracy is 100% because the clotting agent is pressed against or otherwise delivered to the situs immediately before, during, or immediately after the removal of the intrusive device.

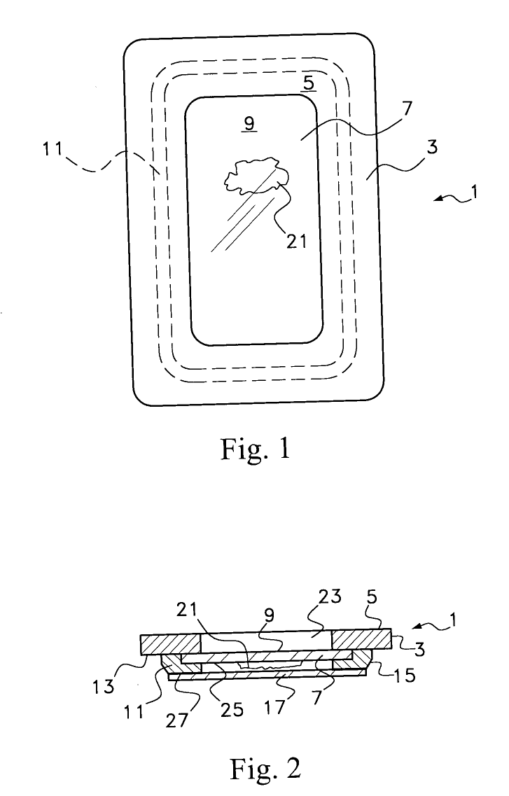

FIG. 1 illustrates a top view and FIG. 2 illustrates a front cut view of one embodiment of the present invention window dressing 1. In each of these two figures, identical parts are identically numbered, and these figures shall be discussed herein collectively. Window dressing 1 includes a top element 3, a middle element 7, a bottom element 15 and a peelable waterproof protective sealing strip 17. Top element 3 has a top 5 and a bottom 13, with a cut out 23, exposing middle element 7, as shown.

Middle element 7 is a clear, water-impermeable plastic window that is attached to the bottom 13 of top element 3. It is larger than the cut out 23 and of compatible configuration (of a shape that fully fits over the window opening, viz., cut out 23). Hence, the perimeter distance around the middle element 7 is less than the outer (first) perimeter distance of top element 3, and greater than the inner (second) perimeter distance of top element 3. On bottom 25 of middle element 7 is a glob of clotting agent 21 that is clearly visible from the top view by a user. The clotting agent could be a plurality of globs, or spread out over part or over the entire exposed underside of middle element 7.

Bottom element 11 is attached to both the bottom of top element 3 and the bottom of middle element 7, as shown. Bottom element 11 also has a cut out that, minimally, overlaps at least a part of cut out 23 of top element 3 to create a window. It is also includes an adhesive layer 27 for attachment to the skin during use. on the bottom 15 of bottom element 11 is a removable (peelable) sealing strip 17, that is pulled off immediately before attachment to the skin. While in place, strip 17 acts as both a protective cover for the adhesive used to attach the device to the skin, and as a seal to preserve sterility of the inside of the window and the clotting agent.

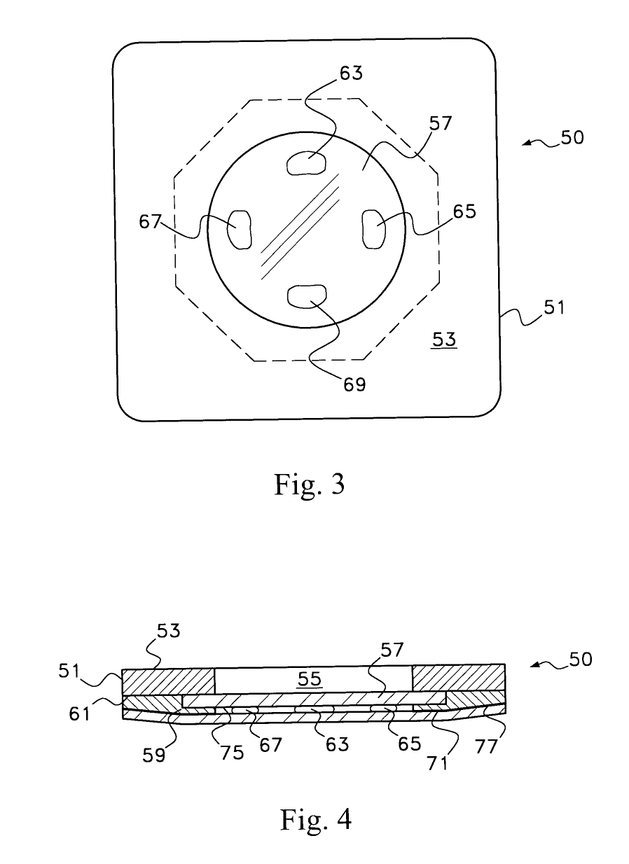

FIG. 3 and FIG. 4 show top and front cut views of an alternative embodiment present invention clotting agent-containing window dressing 50. In each of these two figures, identical parts are identically numbered, and these figures shall be discussed together. Window dressing 50 is a square, flat plastic-coated gauze sheet that includes a top element 51, a middle element 57, a bottom element 59 and a peelable waterproof protective sealing strip 71. Top element 51 has a top 53 and a bottom 61, with a cut out 55, exposing middle element 57, as shown.

Middle element 57 is a clear, water-impermeable, octagonal plastic window that is attached to the bottom 61 of top element 51, appearing as a clear circular window in top element cut out 55. It is larger than the cut out 55 so as to fully cover cut out 55, and the perimeter distance around the middle element 57 is less than the outer (first) perimeter distance of top element 51, and greater than the inner (second) perimeter distance of top element 51. Stuck to bottom 75 of middle element 57 are a four separate deposits of clotting agent 63, 65, 67 and 69, and these are clearly visible from the top view by a user.

Bottom element 59 is attached to both the bottom of top element 51 and the bottom of middle element 57, as shown. Bottom element 59 also has a cut out that, in this embodiment perfectly coincides with cut out 55 of top element 51 to create the window. It is also includes an adhesive layer 77 for attachment to the skin during use. Attached thereto, as shown, is a removable (peelable) sealing strip 71, that is pulled off immediately before attachment to the skin. While in place, strip 71 acts as a protective cover for the adhesive used to attach the device to the skin, and as a seal to preserve sterility of the inside of the window and the clotting agent.

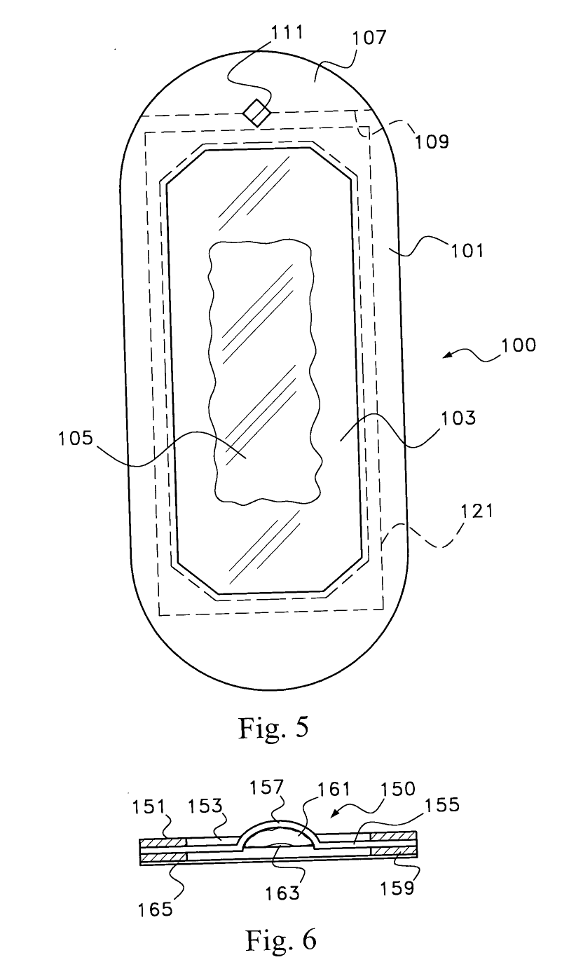

FIG. 5 illustrates a top view of yet another alternative embodiment present invention clotting agent-containing window dressing 100. Window dressing 100 includes a top element 101, a middle element 103, and a bottom element 121. Middle element 103 is fitted between the cut out of top element 101 and the cut out of bottom element 121, and attached to both. Under the middle element 103 is a large area of clotting agent 105. The bottom of top element 101 includes an adhesive (not shown) for attachment to skin. A sealing strip on the bottom of the device may be included in a fashion similar to those described above in conjunction with the preceding Figures.

In this embodiment, top element 101 includes an invasive device insertion port or cut out 111. Perforatedly connected to top element 101 is shroud collar 107. This may include adhesive on its back, and a peelable strip thereon.

A doctor or nurse would lee off the back strip and apply it to the subject's skin at the appropriate location. Shroud collar 107 could be removed (torn off) from the window dressing before or after the it is affixed to the subject. A needle or other invasive device is inserted at cut out 11, and the collar 107 is wrapped around the invasive device (at least partially) and adhered to the dressing and possibly part of the skin, to seal and help hold the invasive device. The needle or other invasive device could be seen through the window, and is then injected at the correctly selected and viewed situs. When the treatment is completed, the doctor or nurse may simultaneously remove the invasive device, and move or push the clotting agent to the location of the invasive device to immediately initiate clotting, long before the window dressing is removed.

FIG. 6 shows a side cut view of another embodiment of the present invention cutting agent-containing window dressing 150. It includes a top element 151 with cut out 153, middle element 155 with semiridge dome 157 and framed bottom element 159. Clotting agent 161 is within dome 157 and has its own small cover 163. Peelable strip 165 seals and protects the underside and is removably attached to an adhesive on the bottom of bottom element 159.

Obviously, numerous modifications and variations of the present invention are possible in light of the above teachings. It is therefore understood that within the scope of the appended claims, the invention may be practiced otherwise than as specifically described herein.

1. A window dressing with a blood clotting agent, which comprises:
a.) a top element, said top element having a top and a bottom and being of a predetermined shape with a first perimeter distance, and having a central cut-out area with a second predetermined distance, said first perimeter distance being greater than said second perimeter distance;
b.) a middle element, said middle element having a top and a bottom, and being a clear polymer window and having a predetermined size, shape, and window perimeter distance greater than said second perimeter distance, said middle element being located beneath said top element and being attached to said bottom of said top element.
c.) a bottom element having a top and a bottom and having a central cut out area, said bottom element central cut out area and said top element central cut out area being at least partially coincidental, said top of said bottom element being attached to at least one of said bottom of said top element and said bottom of said middle element; and
d.) a clotting agent attached to said bottom of said middle element and within said cut out of said bottom element.

2. The window dressing with a blood clotting agent of claim 1 wherein said clotting agent is in a gel format.

3. The window dressing with blood clotting agent of claim 1 wherein said bottom of said bottom element includes an adhesive.

4. The window dressing with blood clotting agent of claim 1 wherein said bottom element has a bottom element perimeter distance that is less than said first perimeter distance of said top element.

5. The window dressing with blood clotting agent of claim 4 wherein said bottom element is centrally positioned relative to said top element and is shaped to establish a bottom inside frame edge of said bottom of said top element.

6. The window dressing with blood clotting agent of claim 5 wherein said bottom element is a blood absorbing material.

7. The window dressing with blood clotting agent of claim 5 wherein said bottom of said top element has adhesive thereon.

8. The window dressing with blood clotting agent of claim 1 wherein said clotting agent includes at least one clotting activator selected from the group consisting of diatomaceous earth, inorganic silicates, ellagic acid, epinephrine and thromboplastin.

9. The window dressing with blood clotting agent of claim 1 wherein said clotting agent is contained within a breakable container and said breakable container is attached to said bottom of said middle element.

10. The window dressing with blood clotting agent of claim 9 wherein said breakable container is a gel cap.

11. The window dressing with blood clotting agent of claim 1 wherein said window dressing further includes at least one invasive device insertion cut out adapted to at least partially enshroud an inserted invasive device.

12. The window dressing of claim 11 wherein said window dressing further includes an invasive device shroud collar for attachment around said invasive device insertion cut out.

13. The window dressing of claim 12 wherein said shroud collar is removably attached to said top element.

14. The window dressing of claim 12 wherein said shroud collar includes an adhesive layer for attachment around said invasive device insertion cut out.

15. The window dressing of claim 3 wherein said dressing further includes a removable sealing strip attached to said adhesive on said bottom of said bottom element.

16. The window dressing of claim 7 wherein said dressing further includes a removable sealing strip attached to said adhesive on said bottom of said top element.

17. The window dressing of claim 15 wherein said removable sealing strip is a waterproof strip that is positioned on said window dressing to cover and seal the bottom of said middle element and said blood clotting agent.

18. The window dressing of claim 16 wherein said removable sealing strip is a waterproof strip that is positioned on said window dressing to cover and seal the bottom of said middle element and said blood clotting agent.

19. The window dressing of claim 1 wherein at least one of said top element and said bottom element is a plastic foam material.

20. The window dressing of claim 1 wherein said middle element is a water impermeable clear polymer material.
